(19) United States
(12) Patent Application Publication
(10) Pub. No.: US 2024/0156868 A1
(43) Pub. Date: May 16, 2024

(54) RECOMBINANT TIM-4 PROTEIN, CHIMERIC ANTIGEN RECEPTOR (CAR) T CELL DELIVERY SYSTEM AND METHODS OF MAKING AND USING SAME

(71) Applicant: Duke University, Durham, NC (US)

(72) Inventors: Peter Fecci, Durham, NC (US); Daniel Wilkinson, Durham, NC (US)

(21) Appl. No.: 18/511,825

(22) Filed: Nov. 16, 2023

Related U.S. Application Data
(63) Continuation of application No. PCT/US2023/065619, filed on Apr. 11, 2023.
(60) Provisional application No. 63/329,551, filed on Apr. 11, 2022.

(51) Int. Cl. A61K 35/17; A61K 39/00; A61P 35/00; C07K 14/705; C07K 16/28; C12N 15/86; G01N 33/574

(52) U.S. Cl. CPC A61K 35/17; A61K 39/4611; A61P 35/00; C07K 14/70503; C07K 16/2809; C12N 15/86; G01N 33/574; A61K 2239/15; C07K 2319/02; C07K 2319/30

(57) ABSTRACT

The present invention provides recombinant TIM-4 fusion proteins comprising a domain of TIM-4 and a scFv specific for CD3 and methods of making and using the same. The fusion proteins provided herein may be administered in combination with CAR T cells. In addition CAR T cells engineered to express the TIM-4 fusion proteins described herein are also provided and may be used in the methods described herein.

### CROSS-REFERENCE TO RELATED APPLICATIONS

This application claims priority to U.S. Provisional Application No. 63/329,551 filed on Apr. 11, 2022, the contents of which are incorporated by reference in their entireties.

### REFERENCE TO AN ELECTRONIC SEQUENCE LISTING

This application includes a sequence listing in XML format titled “155554.00691.xml”, which is 36,976 bytes in size and was created on Apr. 10, 2023. The sequence listing is electronically submitted with this application via Patent Center and is incorporated herein by reference in its entirety.

### BACKGROUND

Immune-based therapies, such as immune checkpoint blockade (ICB) and T cell-redirecting strategies like chimeric antigen receptor (CAR) T cells have garnered excitement over the past couple of decades due to their success in certain subsets of cancers. However, success against solid tumors has been limited, a shortcoming often attributed to solid tumor antigenic heterogeneity. Broadening the repertoire of cancers in which immunotherapies are efficacious remains a major goal of cancer immunology groups worldwide. A multi-specific fusion protein consists of two domains: one domain confers specificity to a tumor antigen and the other domain consists of an anti-CD3 single-chain variable fragment (scFv) that activates T cells through their endogenous T cell receptor. Thus, a multi-specific fusion protein redirects T cells to a selected tumor antigen, independent of T cell receptor specificity. T cell-redirecting therapies are successful when all tumor cells express the same targetable antigen. For example, CAR T cells and multi-specific fusion proteins have revolutionized the treatment of B cell cancers due to the near universal expression of CD19 by malignant cells. Unfortunately, T cell-redirecting therapies have fared poorly in the context of solid tumors. These failures are often attributable to the prolific antigenic heterogeneity displayed by solid tumors.

Accordingly, there is a remaining need in the art for a novel means for identifying and targeting tumor cells. As described in this application, the inventors were able to think outside the box and relied on unconventional, non-protein targets to bypass challenges conferred by tumor heterogeneity.

### SUMMARY

Described herein are TIM-4 fusion proteins composed of a TIM-4 domain linked to a human anti-CD3 scFv and methods of making and using the same. One aspect of the present disclosure provides a TIM-4 fusion protein comprising a TIM-4 domain linked to a single chain variable fragment (scFv) specific for CD3, wherein the TIM-4 domain comprises SEQ ID NO: 3 or a sequence having 95% identity to SEQ ID NO: 3, and wherein the scFv specific for CD3 comprises complementarity determining regions (CDR) of SEQ ID NOs: 22-27 or a sequences with at least 95% identity to SEQ ID NO: 22-27. In some embodiments the scFv specific for CD3 comprises SEQ ID NO: 5, or sequences with at least 95% identity to SEQ ID NO:5. Some embodiments provide a pharmaceutical composition comprising a recombinant TIM-4 fusion protein described herein and a pharmaceutically acceptable excipient, carrier and/or diluent. Some embodiments provide a construct comprising a polynucleotide sequence encoding the TIM-4 fusion protein described herein. Some embodiments provide a chimeric antigen receptor (CAR)-T cell comprising the construct provided herein.

Another aspect of the present disclosure provides a method for treating cancer comprising administering a therapeutically effective amount of a recombinant TIM-4 fusion protein described herein and a pharmaceutically acceptable excipient, carrier and/or diluent. In some embodiments, the method further comprises administering T lymphocytes with the TIM4 fusion protein, wherein T lymphocytes are activated upon binding to the TIM-4 fusion protein. In some embodiments, the T lymphocytes are CAR T cells. In still a further embodiment, the CAR T cells may be engineered to express the TIM4 fusion protein as well as the CAR. In some embodiments, the cancer is an PS-associated cancer or a EGFR-associated cancer.

Another aspect of the present disclosure provides a method of diagnosing cancer comprising administering the fusion protein described herein, and detecting the presence or accumulation of a tag in a subject suspected of having cancer. The tag allows for in vivo detection of the cancer. The TIM4 fusion protein may also be used for in vitro detection of cancer in a biopsy or other sample derived from a subject suspected of having cancer by detecting binding of TIM4 to cells using the tag.

Another aspect of the present disclosure provides a method of inducing a T-cell response in a subject suffering from cancer comprising administering to the subject a therapeutically effective amount of the fusion protein or pharmaceutical composition described herein, wherein an antitumor response to the cancer is induced.

### BRIEF DESCRIPTION OF THE DRAWINGS

FIG. 1. TIM-4 binds glioma and non-glioma tumor cells. Murine glioma (CT-2A, SMA-560), murine non-glioma (B16F10, Lewis lung carcinoma), and human glioma (U87) cells were stained with APC-conjugated recombinant TIM-4.

FIG. 2. Irradiation increases TIM-4 binding to CT-2A glioma cells. A. In vitro cultured CT-2A cells were exposed to 10 Gy radiation and assayed for TIM-4 binding at 0 h and 24 h post-irradiation. B,C. 10⁴ CT-2A glioma cells were implanted intracranially in C57BL/6 mice. After 14 days, mice underwent 10 Gy total body irradiation or were left non-irradiated. 48 hours later, tumors were explanted and stained with TIM-4-APC. Representative overlapping histograms of TIM-4-APC binding to CT-2A is shown in B and the mean fluorescence intensity (MFI) of TIM-4-APC is shown in C.

FIG. 3. Recombinant TIM-4 fusion protein schematic and sequence. A. Model depicting the tumor and T cell binding domains of a recombinant TIM-4 fusion protein. B. The amino acid sequence of a recombinant TIM-4 fusion protein. The fusion protein consists of a signal sequence (SEQ ID NO: 2), the extracellular domain of murine TIM-4 (SEQ ID NO: 7) linked (SEQ ID NO: 3) to a scFv derived from the anti-human CD3 antibody OKT3 (SEQ ID NO: 5) and a tag (SEQ ID NO: 28).

FIG. 4. Recombinant TIM-4 fusion protein binds tumor cells and human T cells. A. U87 tumor cells were incubated with recombinant TIM-4 fusion protein expression supernatant or Mock expression supernatant for 30 minutes. Cells were washed extensively before incubation with Biotinylated Protein L. After further extensive washing, cells were incubated with PE-labelled Streptavidin and then analyzed by flow cytometry. B, C. Human (B) or mouse (C) CD3⁺ T cells were incubated with recombinant TIM-4 fusion protein expression supernatant or Mock expression supernatant for 30 minutes followed by incubation with anti-mouse TIM-4-PE.

FIG. 5. Recombinant TIM-4 fusion protein mediates potent tumor cytotoxicity in vitro. 100,000 human T cells were co-cultured with 10,000 CellTrace Violet-labelled U87 (A) or a 50:50 mix of 5,000 CTV-labelled U87 and 5,000 CTV-labelled U87vIII (B) tumor cells. Recombinant TIM-4 fusion protein alone or EGFRvIII-anti CD3 scFv was titrated into the co-culture. After 24 hours, viable tumor cells were counted and normalized to “No fusion protein” controls to give a percent tumor survival.

FIG. 6. Recombinant TIM-4 fusion protein in combination with EGFRvIII CAR T cells extends survival of mice bearing heterogeneous tumor. NOD-SCID-gamma (NSG) mice were implanted intracranially with 25,000 cells of an 80:20 mix of U87 and U87vIII tumor cells. 3 days later, 2×10⁶ VIII CAR or VIIICAR that secreted TIM-4 fusion protein were administered intracranially, or mice were left untreated. Mice were followed for survival. *p<0.05

FIG. 7. An EGFRvIII CAR modified to secrete recombinant TIM-4 fusion protein extends survival in mice bearing EGFRvIII heterogeneous tumors. A 50:50 mix of CT2A:CT2AvIII was administered IC to C57BL/6 mice. 7 days later, 2×10⁶ VIII CAR or VIIIxTIM-4 CAR were administered to the tumor site. Important note: there was no lymphodepletion preconditioning performed prior to CAR administration.

### DETAILED DESCRIPTION

Immune-based therapies, such as immune checkpoint blockade and T cell-redirecting strategies like chimeric antigen receptor (CAR) T cells have garnered excitement over the past couple of decades due to their success in certain subsets of cancers. However, success against solid tumors has been limited, a shortcoming often attributed to solid tumor antigenic heterogeneity. Novel means for identifying and targeting tumor cells that think outside the box and rely on unconventional, non-protein targets to bypass challenges conferred by tumor heterogeneity are needed. Described herein are TIM-4 fusion proteins composed of a TIM-4 domain linked to a human anti-CD3 scFv and methods of making and using the same.

### Fusion Proteins and Constructs:

One aspect of the present invention provides a recombinant TIM-4 fusion protein comprising a TIM-4 domain linked optionally via a linker to a single chain variable fragment (scFv) specific for CD3. As used herein, a “fusion protein” may also be called a chimeric protein and are proteins created through the joining of two or more genes or portions thereof that originally encoded for separate proteins. Translation of the fusion gene results in a single or multiple polypeptides with functional properties derived from each of the original proteins. Fusion proteins may occur in the body by transfer of DNA between chromosomes or be made recombinantly by combining genes or parts of genes such that a single transcript is formed encoding both polypeptides in frame from the same or different organisms.

T cell membrane protein 4, or TIM-4 (also may be known as T cell membrane protein 4 or T-cell immunoglobulin and mucin domain containing 4) is a protein in humans that is encoded by the TIMD4 gene. Tim-4 is a phosphatidylserine (PS) receptor that is expressed on various immune cell and macrophage subsets. PS is a phospholipid typically present on the cytoplasm-facing side of the plasma membrane, where it remains out of view to the immune system. Cellular stresses can lead to dysregulation of processes that keep PS facing internally and instead promote PS exposure on the cell surface. Tumor cells frequently lose the capacity to regulate their plasma membrane and exhibit detectable levels of PS on their surface, as may normal cells undergoing apoptosis. PS can have a tolerizing effect on the immune system when exposed on the cell surface. While PS exposure thus can be tumor-adaptive, it also leaves the tumor with an “Achilles Heel” that can be targeted immunologically or otherwise. For instance, TIM family proteins bind PS with varying affinity, the strongest binder being TIM-4.

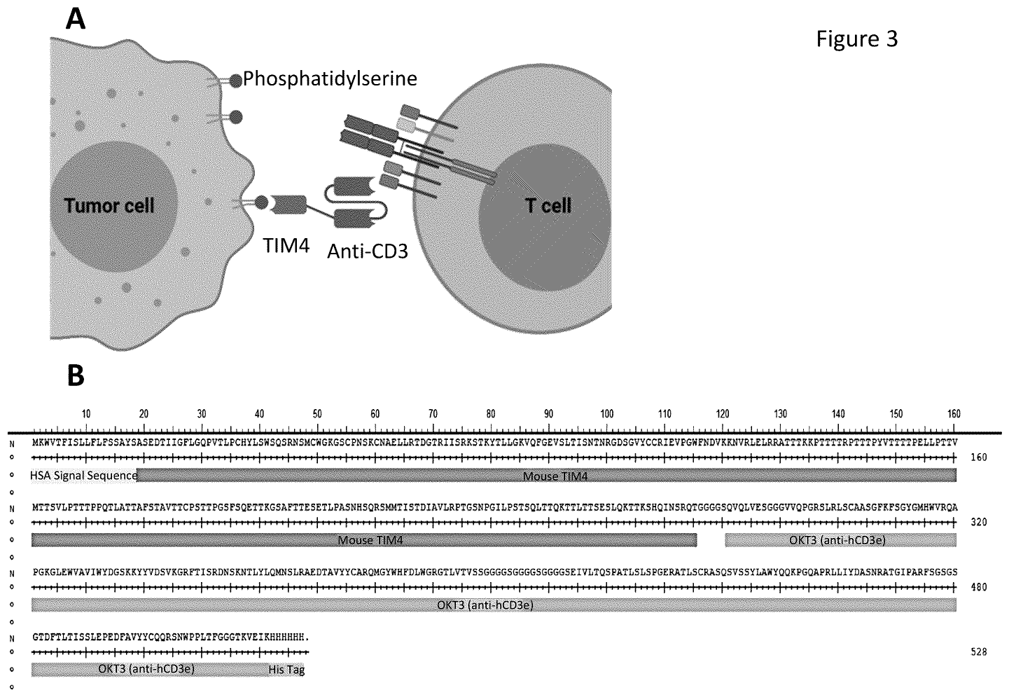

The recombinant TIM-4 fusion protein of the present disclosure comprises a TIM-4 domain comprising SEQ ID NO: 3 or a sequence having 95% identity to SEQ ID NO: 3 and capable of binding to PS. The recombinant TIM-4 fusion protein may be linked via a linker to a scFv specific for CD3. A peptide linker is a short peptide sequence that may be added to link protein domains. Linkers are often composed of flexible residues like glycine and serine so that the adjacent protein domains are free to move relative to one another. Linkers can be varied in length and content as necessary. In some embodiments the linker may comprise glycine and/or serine amino acids in combination. The linker may comprise SEQ ID NO: 4 or any number of continuous repeats of SEQ ID NO:4 or portions thereof. The linker of SEQ ID NO: 4 was used in the Examples to generate the TIM4 fusion protein shown in FIG. 3B. SEQ ID NO: 4 may thus be used to link the TIM4 domain of SEQ ID NO: 3 to the CD3 scFv to generate a TIM4 fusion protein specific for use in humans.

CD3 (cluster of differentiation 3) is a protein complex and T cell co-receptor that is involved in activating both the cytotoxic T cells (CD8+ naive T cells) and T helper cells (CD4+ naive T cells). It is composed of four distinct chains. In mammals, the complex contains a CD3γ chain, a CD3δ chain, and two CD3ε chains. These chains associate with the T-cell receptor (TCR) and the CD3-zeta (ζ-chain) to generate an activation signal in T lymphocytes. The recombinant TIM-4 fusion protein of the present disclosure may be linked via a linker to a single-chain variable fragment (scFv) specific for CD3. A scFv is a fusion protein of the variable regions of the heavy (VH) and light chains (VL) of immunoglobulins, connected with a short linker. The scFv specific for CD3 may comprise complementarity determining regions (CDR) of SEQ ID NO: 22 (VH CDR1), SEQ ID NO: 23 (VH CDR2), SEQ ID NO: 24 (VH CDR3), SEQ ID NO: 25 (VL CDR1), SEQ ID NO: 26 (VL CDR2), and SEQ ID NO: 27 (VL CDR3) or sequences with at least 95% identity to SEQ ID NO: 22-27. The anti-CD3 scFv of the present disclosure may comprise SEQ ID NO: 5 or a sequence with at least 95% identity to SEQ ID NO: 5. The scFv specific for CD3 is capable of binding to and activating T cells via CD3.

The TIM-4 fusion protein may further comprise an N-terminal signal sequence. The N-terminal signal sequence may comprise a secretion signal sequence. A secretion signal sequence or peptide comprises a short peptide present at the N-terminus of proteins that are destined toward the secretory pathway. These proteins include those that reside either inside certain organelles, secreted from the cell, or inserted into cellular membranes. Signal sequences may be removed or cleaved off from the fusion protein. Thus, when administered as a protein, the TIM4 fusion protein will generally lack the signal sequence, but the signal sequence may be needed to allow for adequate expression and secretion of the protein. The nucleotide sequence encoding the protein will generally encode for the signal sequence. In some embodiments, the secretion signal sequence includes a signal sequence of SEQ ID NO: 2 or sequences with at least 90% identity to SEQ ID NO: 2. Signal sequences are generally 15-22 amino acids in length and those of skill in the art will appreciate that one signal sequence can be removed and another signal sequence used in its place.

The fusion proteins provided herein may additionally comprise a tag. The tag may by attached to the protein for various purposes. The tag may comprise any known tag in the art and may be used for the purposes of purification, tracking or imaging the fusion protein. Types of tags may comprise an affinity tag, a solubilization tag, chromatography tag, epitope tag, fluorescent tag or enzymatic modification tag. In some embodiments, the tag may comprise a His tag, a FLAG tag, a fluorescent tag and a Myc tag. In the Examples, a His tag was used.

The fusion protein may comprise a TIM4 domain, a linker and a CD3 scFv. The TIM4 domain and the CD3 scFv may be in any order relative to each other. For example the TIM4 domain may be on the N-terminus of the fusion protein and the CD3 scFv may be on the C-terminus. Alternatively, the CD3 scFv may be on the N-terminis and the TIM4 domain may be on the C-terminus. For example, the fusion protein may comprise SEQ ID NO: 3, 4 and 5 or sequences with at least 95% identity to SEQ ID NO: 3, 4 and 5, wherein SEQ ID NO: 3 and SEQ ID NO: 5 are linked in any order relative to each other via SEQ ID NO: 4. The fusion protein may comprise SEQ ID NO: 2, 3, 4 and 5 or sequences with at least 95% identity to SEQ ID NO: 2, 3, 4 and 5. The fusion protein may comprise SEQ ID NO: 1. The fusion protein may comprise sequences with at least 95% identity to SEQ ID NO: 3, 4 and 5 and a tag, or sequences with at least 95% identity to SEQ ID NO: 2, 3, 4 and 5 and a tag, or sequences with at least 95% identity to SEQ ID NO: 1 and a tag.

In an alternative embodiment, the recombinant TIM-4 fusion protein may comprise an antibody moiety or scFv specific for PS. In some embodiments, the antibody moiety specifically binds to PS present on the surface of a cell. The TIM4 or other region capable of binding to PS binds of PS specifically when PS is found on the surface of a cell. The TIM4 domain or region capable of binding PS does not significantly bind normal, healthy cells that do not generally express PS on their surface. The TIM4 region of the fusion protein is capable of binding to a cell, when the cell is a cancer cell in which PS is not properly regulated and is found on the extracellular side of the cell membrane. The cancer cell with dis-regulated localization of PS may be in a solid tumor, such as a metastatic cancer cell.

The TIM-4 fusion protein may comprise the scFv specific for CD3 or another antigen binding fragment specific for CD3. For example, an antigen-binding fragment selected from the group consisting of a Fab, a Fab′, a F(ab′)2, an Fv fragment, a disulfide stabilized Fv fragment (dsFv), and a single-chain antibody molecule (scFv). In some embodiments, the recombinant TIM-4 fusion protein comprises a scFv specific for CD3. In some embodiments the scFv specific for CD3 comprises complementarity determining regions (CDR) of SEQ ID NO: 22 (VH CDR1), SEQ ID NO: 23 (VH CDR2), SEQ ID NO: 24 (VH CDR3), SEQ ID NO: 25 (VL CDR1), SEQ ID NO: 26 (VL CDR2), and SEQ ID NO: 27 (VL CDR3). In some embodiments the scFV specific for CD3 comprises SEQ ID NO: 5. The recombinant TIM-4 fusion protein may be human, humanized, or semi-synthetic.

The present disclosure provides pharmaceutical compositions comprising one or more of the compositions or fusion proteins as described herein and an appropriate carrier, excipient or diluent. The exact nature of the carrier, excipient or diluent will depend upon the desired use for the composition and may range from being suitable or acceptable for veterinary uses to being suitable or acceptable for human use. The composition may optionally include one or more additional compounds.

When used to treat or prevent a disease or symptoms of a disease, such as cancer, the compositions described herein may be administered singly, as mixtures of one or more compounds or in mixture or combination with other agents (e.g., therapeutic agents) useful for treating such diseases and/or the symptoms associated with such diseases. The compounds may be administered in the form of compounds (i.e. proteins) per se, or as pharmaceutical compositions comprising a compound.

Pharmaceutical compositions comprising the compositions may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compositions into preparations which can be used pharmaceutically. Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

The compositions described herein will generally be used in an amount effective to achieve the intended result, for example in an amount effective to treat or prevent cancer. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized. Treating cancer includes, but is not limited to, reducing the number of cancer cells or the size of a tumor in the subject, reducing progression of a cancer to a more aggressive form, reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc.

The amount of composition administered will depend upon a variety of factors, including, for example, the particular indication being treated, the mode of administration, whether the desired benefit is prophylactic or therapeutic, the severity of the indication being treated and the age and weight of the patient, the bioavailability of the particular composition, the conversion rate and efficiency of delivery under the selected route of administration, etc.

Determination of an effective dosage for a particular use and mode of administration is well within the capabilities of those skilled in the art. Effective dosages may be estimated initially from in vitro activity and metabolism assays. For example, an initial dosage for use in animals may be formulated to achieve a circulating blood or serum concentration of the composition that is at or above an IC₅₀ of the particular composition as measured in an in vitro assay. Calculating dosages to achieve such circulating blood or serum concentrations taking into account the bioavailability of the particular composition via the desired route of administration is well within the capabilities of skilled artisans. Initial dosages can also be estimated from in vivo data, such as animal models. Animal models useful for testing the efficacy of the active metabolites to treat or prevent the various diseases described above are well-known in the art. Animal models suitable for testing the bioavailability and/or metabolism of compositions are also well-known. Ordinarily skilled artisans can routinely adapt such information to determine dosages suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active composition, the bioavailability of the composition, its metabolism kinetics and other pharmacokinetic properties, the mode of administration and various other factors, discussed above. Dosage amount and interval may be adjusted individually to provide plasma levels which are sufficient to maintain therapeutic or prophylactic effect. For example, the compositions may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compositions may not be related to plasma concentration. Skilled artisans will be able to optimize effective dosages without undue experimentation.

The dose of the recombinant TIM-4 fusion protein administered to an individual (such as a human) may vary with the particular composition, the mode of administration, and the type of disease being treated. In some embodiments, the amount of the composition is effective to result in an objective response (such as a partial response or a complete response). In some embodiments, the amount of a recombinant TIM-4 fusion protein in the composition is included in a range of, e.g., about 0.001 pg to about 1000 pg. In some embodiments of any of the above aspects, the effective amount of a recombinant TIM-4 fusion protein in the composition is in the range of about 0.1 pg/kg to about 100 mg/kg of total body weight.

The recombinant TIM-4 fusion protein can be administered to an individual (such as human) via various routes, including, for example, intravenous, intra-arterial, intraperitoneal, intrapulmonary, oral, inhalation, intravesicular, intramuscular, intra-tracheal, subcutaneous, intradermal, intraocular, intrathecal, transmucosal, and transdermal. In some embodiments, sustained continuous release formulation of the composition may be used.

In some embodiments a construct comprising a polynucleotide sequence encoding any recombinant TIM-4 fusion protein described herein is provided. The term “construct” or “polynucleotide construct” is a polynucleotide, either DNA or RNA, which allows the encoded sequence to be replicated and/or expressed in the target cell. A construct may contain an exogenous promoter, operably linked to any one of the polynucleotides described herein. As used herein, a polynucleotide is “operably connected” or “operably linked” when it is placed into a functional relationship with a second polynucleotide sequence. The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease Si), as well as protein binding domains (consensus sequences) responsible for the binding of RNA polymerase.

In some embodiments, the construct is in an expression construct, a vector or a viral vector. A vector is any particle used as a vehicle to artificially carry a foreign nucleic sequence, typically DNA into another cell, where it can be replicated and/or expressed. A vector containing foreign DNA is termed recombinant DNA. The four major types of vectors are plasmids, viral vectors, cosmids, and artificial chromosomes. Expression constructs comprise a heterologous promoter and the nucleic acid sequence encoding protein of interest (e.g., recombinant TIM-4, CD3 scFv) which is capable of expression in the cell in which it is introduced. The expression constructs include vectors which are capable of directing the expression of exogenous genes to which they are operatively linked. Such vectors are referred to herein as “recombinant constructs,” “expression constructs,” “recombinant expression vectors” (or simply, “expression vectors” or “vectors”) and may be used interchangeably. Suitable vectors are known in the art and contain the necessary elements in order for the gene encoded within the vector to be expressed as a protein in the host cell. The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments encoding the mutant α-gal protein. Another type of vector is a viral vector, wherein additional DNA segments may be ligated into the viral genome. Viral vectors are incorporated into viral particles that are then used to transport the viral polynucleotide encoding the protein of interest into the target cells. Certain vectors are capable of autonomous replication in a host cell into which they are introduced. Other vectors can be integrated into the genome of a host cell upon introduction into the host cell, and thereby are replicated along with the host genome (e.g., lentiviral vectors). Moreover, certain vectors are capable of directing the expression of exogenous genes to which they are operatively linked. In general, vectors of utility in recombinant DNA techniques are often in the form of plasmids. In the present specification “vector” include expression vectors, such as viral vectors (e.g., replication defective retroviruses (including lentiviruses), adenoviruses and adeno-associated viruses (AAV)), which serve equivalent functions.

The vectors are heterogeneous exogenous constructs containing sequences from two or more different sources. Suitable vectors include, but are not limited to, plasmids, expression vectors, lentiviruses (lentiviral vectors), adeno-associated viral vectors (rAAV), among others and includes constructs that are able to express the protein of interest in cells. A preferred vector is a lentiviral vector or adeno-associated vector. Suitable methods of making viral particles are known in the art to be able to transform cells in order to express the protein of interest in cells. The cells may be T cells and may be autologous T cells or CAR T cells as described more fully below. The CART cells may be CAR T cells capable of targeting the cancer, such as EGFR vIII CAR T cells which may comprise SEQ ID NO: 10 or D2C7 CAR T cells which may comprise SEQ ID NO: 17. Those of skill in the art can determine other CAR T cells that may be used in combination with the TIM4 fusion proteins and constructs encoding the TIM4 fusion proteins provided herein.

Heterologous promoters useful in the practice of the present invention include, but are not limited to, constitutive, inducible, temporally-regulated, developmentally regulated, chemically regulated, tissue-preferred, tissue-specific promoters and cell-type specific. The heterologous promoter may be a plant, animal, bacterial, fungal, or synthetic promoter. Suitable promoters are known and described in the art. In mammalian cells, typical promoters include, without limitation, promoters for Rous sarcoma virus (RSV), human immunodeficiency virus (HIV-1), cytomegalovirus (CMV), SV40 virus, as well as the translational elongation factor EF-1α promoter or ubiquitin promoter.

In some embodiments a construct comprising a polynucleotide sequence encoding any recombinant TIM-4 fusion protein described herein may further comprise a sequence encoding a chimeric antigen receptor (CAR). The CAR and the TIM4 fusion protein may be expressed using distinct promoters or as described below may be expressed using a single promoter and may be on a single construct or expression vector or may be on separate constructs or expression vectors to transfect or be introduced into T cells. The term “chimeric antigen receptor” or “chimeric receptor” or “CAR” or “CARs” as used herein refers to a polypeptide having a pre-defined binding specificity to a desired target and operably connected to (e.g., as a fusion or as separate chains linked by one or more disulfide bonds, etc.) the intracellular part of a T-cell activation domain. More particularly, CAR are engineered receptors, which, when expressed graft an antigen specificity onto a cytotoxic cell, for example T cells, NK cells or macrophages. For example, CAR proteins are engineered to give T cells the new ability to target a specific protein. The CARs of the present invention may comprise an extracellular domain with at least one antigen specific targeting region, a transmembrane domain (TM), and an intracellular domain (ID) including one or more co-stimulatory domains (CSD) in a combination that is not naturally found together on a single protein. This particularly includes receptors wherein the extracellular domain and the cytoplasmic domain are not naturally found together on a single receptor protein. Further, the chimeric receptor is different from the TCR expressed in the native T cell lymphocyte.

The term “CAR-T cells” as used herein refer to a T cell or population thereof, which has been modified through molecular biological methods to express a chimeric antigen receptor (CAR) on the T cell surface. The CAR is a polypeptide having a pre-defined binding specificity to a desired target expressed operably connected to (e.g., as a fusion, separate chains linked by one or more disulfide bonds, etc.) the intracellular part of a T-cell activation domain. By bypassing MHC class I and class II restriction, CAR engineered T cells of both CD8⁺ and CD4⁺ subsets can be recruited for redirected target cell recognition. The most common CARs are fusions of immunoglobulin binding functionality (e.g., as a single-chain variable fragment (scFv) derived from a monoclonal antibody) to CD3-zeta (CD3ζ) transmembrane and endodomain. Such molecules result in the transmission of a zeta signal in response to recognition by the immunoglobulin binding functionality of its target. There are, however, many alternatives. By way of example, an antigen recognition domain from native T-cell receptor (TCR) alpha and beta single chains may be used as the binding functionality. Alternatively, receptor ectodomains (e.g. CD4 ectodomain) or cytokines (which leads to recognition of cells bearing the cognate cytokine receptor) may be employed. All that is required of the binding functionality is that it binds a given target with high affinity in a specific manner.

The transmembrane domain may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of) the a, b, d, or g chain of the T-cell receptor, CD28, CD3.epsilon., CD3z, CD45, CD4, CD5, CD8. CD9, CD16. CD22, CD33, CD37, CD64, CD80, CD86, CD134, CD137, or CD154. In some embodiments, the transmembrane domain may be synthetic, in which case it may comprise predominantly hydrophobic residues such as leucine and valine. In some embodiments, a triplet of phenylalanine, tryptophan and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of the CAR as provided herein. In some embodiments, the linker is a glycine-serine doublet.

The intracellular signaling domain of the CAR-T cell provided herein is responsible for activation of at least one of the normal effector functions of the immune cell in which the CAR-T cell as provided herein has been placed in. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the CAR-T cell according to the present disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

The present disclosure may comprise a CAR-T cell comprising a construct of any recombinant TIM-4 fusion protein as described herein and a CAR of any specificity. Thus, the TIM-4 recombinant fusion protein may be used in combination with any CAR for which targeting PS is desired. Any recombinant TIM-4 fusion protein described herein may be combined with a CAR in one or more constructs. A recombinant TIM-4 fusion protein may include a self-cleavage site when included in a single construct with a CAR, such as a 2A self-cleaving peptide. The CAR combined with the TIM-4 fusion protein may comprises an extracellular domain comprising an antigen binding region which binds to one or both of a wildtype EGFR and an EGFR VIII variant. The extracellular antigen binding region may comprise a scFv of SEQ ID NO: 10 or sequences with at least 95% identity to SEQ ID NO: 10 or a scFv of SEQ ID NO: 17 or sequences with at least 95% identity to SEQ ID NO: 17. In some embodiments, the CAR and the TIM-4 fusion protein are connected via a self-cleavage site. In some embodiments the self-cleavage sequence may compromise a 2A self-cleaving peptide. In one embodiment the 2A self-cleaving peptide is SEQ ID NO: 11 or sequences with at least 95% identity to SEQ ID NO: 11.

The present disclosure may comprise a CAR-T cell comprising a construct of any recombinant TIM-4 fusion protein as described herein and any CAR as described herein or elsewhere. The CAR-T may comprise a construct comprising a polynucleotide encoding a EGFR VIII CAR of SEQ ID NO: 10, a self-cleavage site, a signal sequence, a TIM-4 domain of SEQ ID NO: 3, a linker, and an scFv specific for CD3 of SEQ ID NO: 22-27 or wherein the construct comprises a polynucleotide encoding a polypeptide with at least 95% identity to SEQ ID NO: 10, 3, 22, 24, 25, 26 and 27. The CAR-T may comprise a construct comprising a polynucleotide encoding a EGFR VIII CAR of SEQ ID NO: 10, a self-cleavage site, a signal sequence, a TIM-4 domain of SEQ ID NO: 3, a linker and an scFv specific for CD3 of SEQ ID NO: 5 or wherein the construct comprises a polynucleotide encoding a polypeptide with at least 95% identity to SEQ ID NO: 10, 3 and 5. In some embodiments the CAR-T may comprise a construct comprising a polynucleotide encoding a D2C7 CAR of SEQ ID NO: 17, a self-cleavage site, a signal sequence, a TIM-4 domain of SEQ ID NO: 3, a linker and an scFv specific for CD3 of SEQ ID NO: 22-27 or wherein the construct comprises a polynucleotide encoding a polypeptide with at least 95% identity to SEQ ID NO 17, 3, 22, 23, 24, 25, 26 and 27. In some embodiments the CAR-T may comprise a construct comprising a polynucleotide encoding a D2C7 CAR of SEQ ID NO: 17, a self-cleavage site, a signal sequence, a TIM-4 domain of SEQ ID NO: 3, a linker and an scFv specific for CD3 of SEQ ID NO: 5 or wherein the construct comprises a polynucleotide encoding a polypeptide with at least 95% identity to SEQ ID NO 17, 3 and 5. The constructs may comprise a self-cleavage site comprising SEQ ID NO: 11 or a sequence with at least 95% sequence identity to SEQ ID NO: 11. The constructs may comprise a signal sequence comprising SEQ ID NO: 2 or a sequence with at least 95% sequence identity to SEQ ID NO: 2. The constructs may comprise a linker of SEQ ID NO: 4.

The present disclosure provides a CAR-T cell comprising a construct encoding a CAR of SEQ ID NO: 10, a self-cleavage site of SEQ ID NO: 11, a signal sequence of SEQ ID NO: 2, a TIM-4 domain of SEQ ID NO: 3, a linker of SEQ ID NO: 4 and an scFv specific for CD3 of SEQ ID NO: 22-27. The present disclosure provides a CAR-T cell comprising a construct encoding a CAR of SEQ ID NO: 10, a self-cleavage site of SEQ ID NO: 11, a signal sequence of SEQ ID NO: 2, a TIM-4 domain of SEQ ID NO: 3, a linker of SEQ ID NO: 4 and an scFv specific for CD3 of SEQ ID NO: 5. The present disclosure provides a CAR-T cell comprising a construct encoding a CAR of SEQ ID NO: 17, a self-cleavage site of SEQ ID NO: 11, a signal sequence of SEQ ID NO: 2, a TIM-4 domain of SEQ ID NO: 3, a linker of SEQ ID NO: 4 and an scFv specific for CD3 of SEQ ID NO: 22-27. The present disclosure provides a CAR-T cell comprising a construct encoding a CAR of SEQ ID NO: 17, a self-cleavage site of SEQ ID NO: 11, a signal sequence of SEQ ID NO: 2, a TIM-4 domain of SEQ ID NO: 3, a linker of SEQ ID NO: 4 and an scFv specific for CD3 of SEQ ID NO: 5. CAR-T cells comprising a construct encoding SEQ ID NO: 20 or a sequence with at least 95% sequence identity to SEQ ID NO: 20 are provided. CAR-T cells comprising a construct encoding SEQ ID NO: 21 or a sequence with at least 95% sequence identity to SEQ ID NO: 21 are also provided.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. These reporter genes may also be used as tags and be encoded as part of the TIM4 fusion protein and be used as a means to ensure the TIM4 protein is being expressed. Suitable reporter genes may include genes encoding luciferase, β-galactosidase, chloramphenicol acetyl transferase, secreted alkaline phosphatase, or the green fluorescent protein gene (e.g., Ui-Tel et al., FEBS Letters, 479, 2000). Several of these reporters have split reporter function where a portion of the reporter can be provided in trans to significantly reduce the size of the tag to effect labeling of the fusion protein. Such split reporters are available commercially and are well known to those of skill in the art. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays.

In some embodiments, sequence variants provided herein are contemplated. For example, it may be desirable to improve the binding affinity and/or other biological properties of the antibody moiety. Amino acid sequence variants of a fusion protein or construct may be prepared by introducing appropriate modifications into the nucleotide sequence encoding the fusion protein, or by peptide synthesis. Such modifications include, for example, deletions from, and/or insertions into and/or substitutions of residues within the amino acid sequences of the antibody moiety. Any combination of deletion, insertion, and substitution can be made to arrive at the final construct, provided that the final construct possesses the desired characteristics, e.g., antigen-binding. Compositions and sequences provided herein, including recombinant TIM-4 fusion proteins may also comprise species specific variations. For example, SEQ ID NOs: 1, 2, 3, 4, 5, 10 and 17 are human sequences and SEQ ID NOs: 7, 8, 14 and 17 are murine sequences. Species specific sequences and variations may be used depending on the desired subject species and outcomes.

### Methods:

A second aspect of the present invention provides a method for treating cancer comprising administering a therapeutically effective amount of any recombinant TIM-4 fusion protein described herein and a pharmaceutically acceptable excipient, carrier and/or diluent.

Treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer. In some embodiments, the optimum effective amount can be readily determined by one skilled in the art using routine experimentation. The aim of treatment includes the alleviation or prevention of symptoms, slowing or stopping the progression or worsening of a disease, disorder, or condition and/or the remission of the disease, disorder or condition. The term “effective amount” or “therapeutically effective amount” refers to an amount sufficient to effect beneficial or desirable biological and/or clinical results.

As used herein, the term “administering” an agent, such as a therapeutic entity to composition described herein an animal or cell, is intended to refer to dispensing, delivering or applying the substance to the intended target. In terms of the therapeutic agent composition, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The methods of the present disclosure can be used to treat any cancer, and any metastases thereof, including, but not limited to, carcinoma, lymphoma, blastoma, sarcoma, and leukemia. In some embodiments, the cancer is characterized by, or associated with phosphatidylserine (PS) expression (herein referred to as an “PS-associated cancer”). PS is a phospholipid that is normally present on the inner leaflet of normal cells. However, apoptotic as well as non-apoptotic cancer cells such as malignant melanoma, leukemia, neuroblastoma, and gastric carcinoma have been shown to widely express PS on their surfaces. PS exposed on the surface of tumor cells contributes to suppression of T-cell activity and blocks tumor clearance. In some embodiments, the cancer comprises those with PS on the cell surface. In some embodiments the cancer comprises EGFR-associated cancers which are those cancer associated with EGFR expression. Suitable examples include, but are not limited to, of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. In some embodiments, the cancer comprises glioblastoma.

In some embodiments the method further comprises administering effector cells, in particular, T lymphocytes, wherein T lymphocytes are activated upon binding to the TIM-4 fusion protein. The T lymphocytes may be autologous or allogeneic or differentiated T cells from a stem or multipotent cell. The T lymphocytes may be nonspecific for a target or antigen. Exemplary effector cells useful for the present disclosure include, but are not limited to, dendritic cells (including immature dendritic cells and mature dendritic cells), T lymphocytes (such as naive T cells, effector T cells, memory T cells, cytotoxic T lymphocytes, T helper cells, Natural Killer T cells, Treg cells, tumor infiltrating lymphocytes (TIL), and lymphokine-activated killer (LAK) cells), B cells, Natural Killer (NK) cells, monocytes, macrophages, neutrophils, granulocytes, and combinations thereof. Subpopulations of effector cells can be defined by the presence or absence of one or more cell surface markers known in the art (e.g., CD3, CD4, CD8, CD19, CD20, CD11c, CD123, CD56, CD34, CD14, CD33, etc.).

T cell activation refers to a process in which mature T cells can express antigen-specific T cell receptors on their surface to recognize their cognate antigens and respond by entering the cell cycle, clonally expand and differentiate, secreting cytokines or lytic enzymes, and initiating the cell-based functions of the immune system. Cytokine release is a consequence of T cell activation and efficacy, it is preferred that at least a portion of the activated T cell produce one or more cytokine such as those selected from the group consisting of IL-1, IL-1β, IL-2, IL-4, IFN-γ, IL-10, IL-12, TNF-α and GM-CSF. Additionally, at least a portion of the activated T cells preferably express one or more surface markers selected from the group consisting of CD2, CD28, CTLA4, CD40 ligand (gp39), CD18, CD25, CD69, CD16/CD56, MEW Class I, MEW Class II, CD8, CD4, CD3/TcR, CD54, LFA-1 and VLA-4. The T cell may be activated in any way known in the art prior to administration. The T lymphocytes may also become activated or specifically activated upon binding to the TIM-4 fusion protein. T lymphocytes may bind to the scFv anti-CD3 domain of the TIM-4 fusion protein. The T lymphocytes may be CAR T cells. The CAR T cells may comprise an EGFR VIII CAR of SEQ ID NO: 10 or a D2C7 CAR of SEQ ID NO: 17 or sequences having at least 95% identity to SEQ ID NO: 10 or SEQ ID NO: 17.

In some embodiments the recombinant TIM-4 fusion protein may be administered separately from the T lymphocyte or CAR-T cell. In some embodiments the recombinant TIM-4 fusion protein may be administered together with T lymphocyte and/or including CAR-T cells. A recombinant TIM-4 fusion protein of the present disclosure may be administered before, together with or following the administration of T lymphocytes, CAR-T cells or any other cancer therapy including other immunotherapies, chemotherapy, radiation, surgery or any other standard of care cancer treatment. The recombinant TIM-4 fusion protein of the present disclosure may be administered with a CAR-T cell of any specificity.

Some embodiments of the present disclosure provide a method for treating cancer comprising administering a therapeutically effective amount of any of the CAR-T cells described herein and a pharmaceutically acceptable excipient, carrier and/or diluent. In some embodiments the cancer is a PS-associated cancer or an EGFR-associated cancer including those described herein. Some embodiments of the present disclosure provide a method for treating cancer comprising administering a cell comprising any construct described herein. In some embodiments the cancer is a PS-associated cancer or an EGFR-associated cancer including those described herein.

Another aspect of the present disclosure provides a method of diagnosing cancer comprising any of the TIM-4 fusion proteins provided herein, detecting the presence or accumulation of a tag in a subject suspected of having cancer wherein the tag allows for in vivo or in vitro by contacting cells from a subject for detection of the cancer. A medical diagnosis is the process of determining which disease or condition explains a person's symptoms and signs. The cancer may be a PS-associated cancer or an EGFR-associated cancer including those described herein.

In some embodiments the present disclosure proves a method of inducing a T-cell response in a subject suffering from cancer comprising administering to the subject a therapeutically effective amount of any of the fusion proteins or pharmaceutical compositions described herein wherein an antitumor response to the cancer is induced.

Anther embodiment of the present disclosure provides a method of inducing a T-cell response in a subject suffering from cancer comprising administering to the subject a therapeutically effective amount of any of the CAR-T cells described herein, wherein an antitumor response to the cancer is induced.

### Additional Definitions

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

As used herein, the term “specifically” or “selectively” binds, when referring to a ligand/receptor, nucleic acid/complementary nucleic acid, antibody/antigen, or other binding pair (e.g., a cytokine to a cytokine receptor) indicates a binding reaction which is determinative of the presence of the protein in a heterogeneous population of proteins and other biologics. Thus, under designated conditions, a specified ligand binds to a particular receptor and does not bind in a significant amount to other proteins present in the sample. Specific binding can also mean, e.g., that the binding compound, nucleic acid ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

“Percentage of sequence identity” or “percent similarity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The methods of treatment of the human body may be reformatted into second medical use or other related claim formats and include use of the compositions provided herein for production of medicaments for use in treatment of cancer or diagnosis of cancer.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

In the following example, the inventors describe a recombinant TIM-4 fusion protein and methods of using the same.

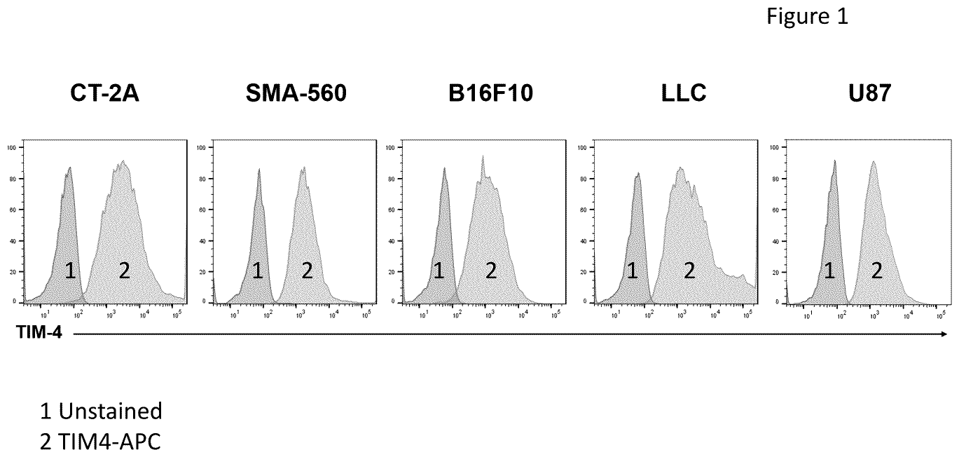

To validate the strategy of targeting PS with TIM-4, it was initially sought to determine the extent of TIM-4 binding to various murine and human tumor cells lines. For these studies, fluorescently tagged recombinant TIM-4 with allophycocyanin (APC) was used in order to detect TIM-4 binding by flow cytometry. Compared to unstained control, TIM-4 bound virtually all tumor cell lines tested (FIG. 1). These tumor cell lines included murine CT-2A and SMA-560 gliomas, murine B16F10 melanoma, murine Lewis lung carcinoma (LLC), and human U87 glioma. Notably, TIM-4 binding overlapped with Annexin V, a known binder of PS, suggesting TIM-4 is indeed binding PS on tumor cells (data not shown). These data provide evidence that TIM-4 is a viable targeting moiety for a variety of cancer types.

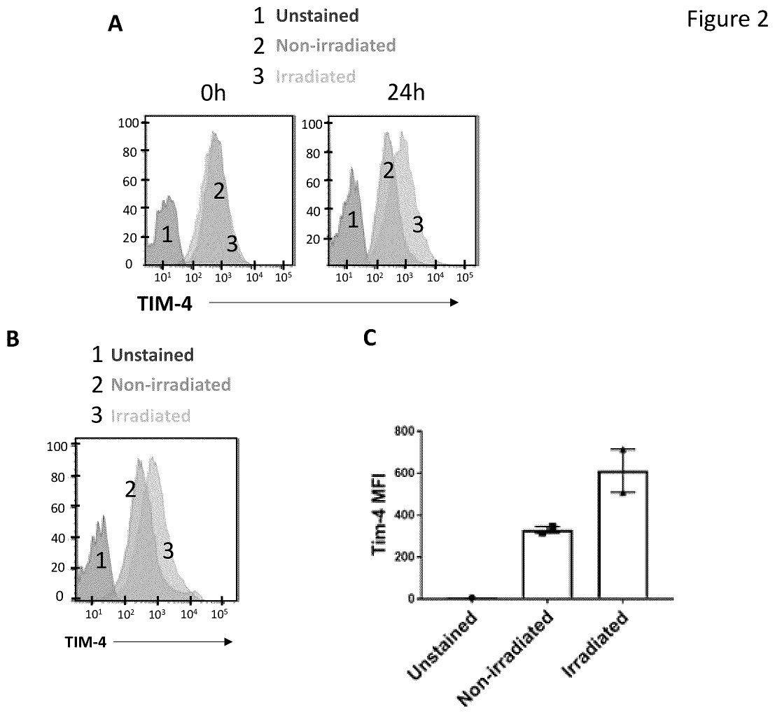

One of the risks involved with any targeted therapy is toxicity. PS exposure is not limited to tumor cells but can also be found on platelets, activated T cells, and apoptotic cells. Therefore, recombinant TIM-4 could potentially induce on-target, off-tumor toxicities. In an effort to limit these toxicities, radiation was used to enhance PS exposure on the surface of tumor cells. This increases the target load, ideally resulting in more tumor-specific killing by the recombinant TIM-4 fusion protein. To test this, CT-2A tumor cells were first irradiated in vitro at a dose of 10 Gy and then seeded into flasks. After 24 hours, cells were removed from culture and stained with APC-conjugated TIM-4. The mean fluorescence intensity (MFI) of TIM-4 binding was considerably higher in the irradiated compared to non-irradiated tumor cells, suggesting an enhancement in PS exposure following irradiation (FIG. 2A). Next, it was sought to determine if PS exposure and TIM-4 binding increased following irradiation of tumors in vivo. To test this, CT-2A was implanted intracranially in C57BL/6 mice. After 14 days, mice were irradiated with Gy total body irradiation or left non-irradiated. After 48 hours, tumors were harvested, disassociated, and stained with APC-conjugated TIM-4. Similarly to the in vitro observations, TIM-4 binding and PS exposure increased on tumor cells following irradiation (FIG. 2B, C). These data provide evidence that radiation, which is already a staple of cancer therapy, enhances PS exposure, and thus should synergize with the TIM-4-targeting strategy according to the present disclosure.

Multi-specific antibody displays potent tumor cytotoxic capabilities in circumstances where tumor antigens are homogeneously expressed. Unfortunately, homogeneous antigen expression is rare, especially in solid tumors. However, based on the data provided herein, PS represents a universal, targetable entity. Therefore, the inventors sought to develop a novel T cell-redirecting therapy based on multi-specific antibody technology that targets surface exposed PS. To this effect, a multi-specific antibody molecule comprising the extracellular domain of TIM-4 covalently linked to an agonist anti-human CD3 scFv was designed and expressed. A schematic depicting the structure of a recombinant TIM-4 fusion protein along with the amino acid sequence of a recombinant TIM-4 fusion protein in accordance with one embodiment of the present disclosure are shown in FIG. 3B.

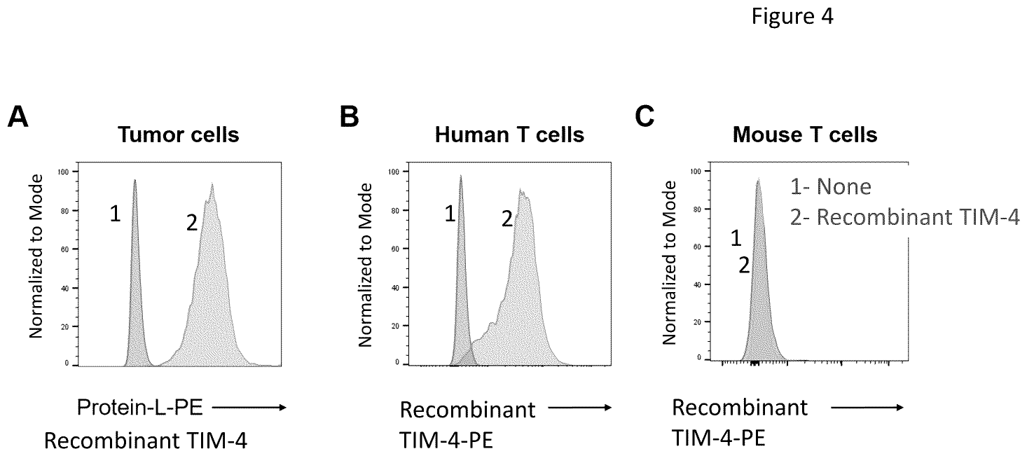

It was first sought to determine if each domain of recombinant TIM-4 fusion protein retains binding to their respective targets. Recombinant TIM-4 fusion protein (human) was incubated with U87 tumor cells followed by detection with Protein-L (which binds the scFv) (FIG. 4A). Recombinant TIM-4 was detectable on U87 cells, suggesting recombinant TIM-4 fusion protein is able to bind tumor cells through PS. Next, recombinant TIM-4 fusion protein was incubated with human T cells followed by detection with a fluorescently-labeled anti-mouse TIM-4 antibody (FIG. 4B). Recombinant TIM-4 was detectable on human T cells, suggesting recombinant TIM-4 fusion proteins can redirect T cells to PS on tumor cells. This T cell binding was specific to human T cells, as recombinant TIM-4 was not detectable after incubation with mouse T cells (FIG. 4C).

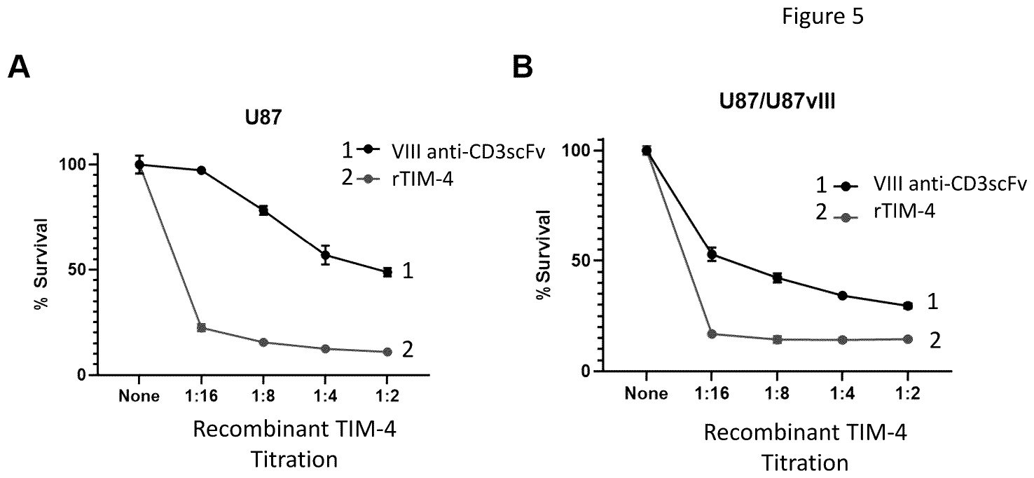

Armed with a recombinant TIM-4 fusion protein, functionality was next tested by determining if recombinant TIM-4 could efficiently mediate killing of tumor cells in in vitro cytotoxicity assays. In these assays, increasing concentrations of human recombinant TIM-4 fusion protein was titrated into co-cultures of human T cells and tumor cells. Tumor cells were labeled with a fluorescent dye to allow for quantification of tumor cells after 24 hours by flow cytometry. By normalizing the number of tumor cells remaining in each condition to the number of tumor cells in the absence of recombinant TIM-4, a percent survival can be calculated. Recombinant TIM-4 demonstrates effective, dose-dependent killing of U87 (FIG. 5A). Importantly, in a heterogeneous tumor population consisting of U87 and U87 transfected with a tumor-specific antigen, EGFRvIII (U87vIII), recombinant TIM-4 was more effective than an EGFR VIII-targeting recombinant TIM-4 (FIG. 5B). These data suggest recombinant TIM-4 mediates potent tumor cytotoxicity, even when tumors demonstrate antigenic heterogeneity. Likewise, this suggests that recombinant TIM-4 is able to accomplish the goal: effective cytotoxicity that persists even in the face of protein antigen heterogeneity—and that they are superior to current platforms in their capacity to do so.

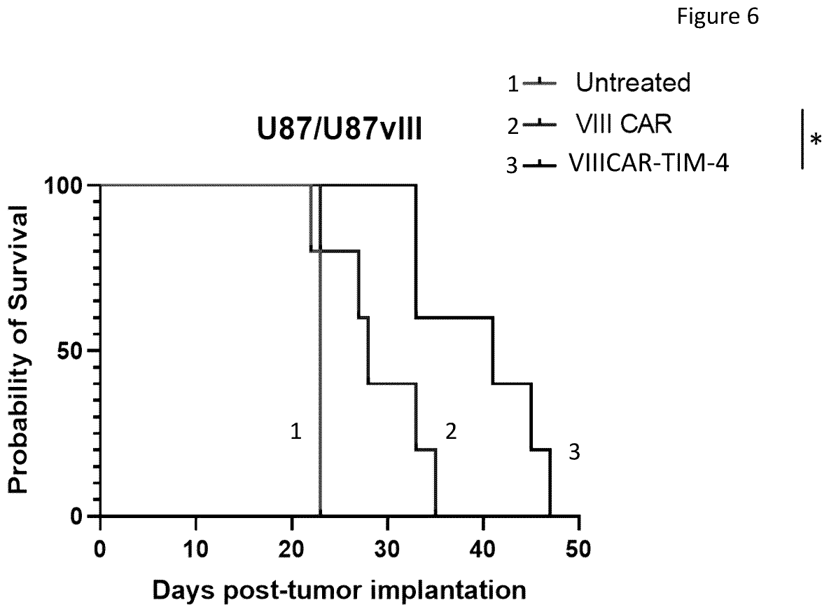

Multi-specific antibodies have relatively short half-lives in vivo which means chronic administration of multi-specific antibodies is necessary to maintain the systemic levels required for efficacy. To circumvent this issue and to decrease the likelihood of on-target, off-tumor toxicity by recombinant TIM-4, the inventors have engineered tumor antigen (EGFRvIII)-specific CAR T cells to serve as a tumor-localizing vehicle that will secrete recombinant TIM-4 fusion protein at the tumor site in renewable fashion. Thus, recombinant TIM-4 fusion protein is delivered locally by a CAR T cell that also possesses its own anti-tumor efficacy, proffering a combinatorial therapy. For proof-of-concept, a VIII-targeting CAR was engineered that secreted recombinant TIM-4. These CAR T cells were then administered intracranially to mice bearing a heterogeneous tumor consisting of U87 and U87vIII cells (FIG. 6). Compared to VIII CAR alone, VIII CAR secreting recombinant TIM-4 significantly prolonged survival of mice. These data provide evidence for in vivo efficacy of recombinant TIM-4, as well as for the use of CAR T cells secreting recombinant TIM-4 as an effective delivery vehicle for the therapy.

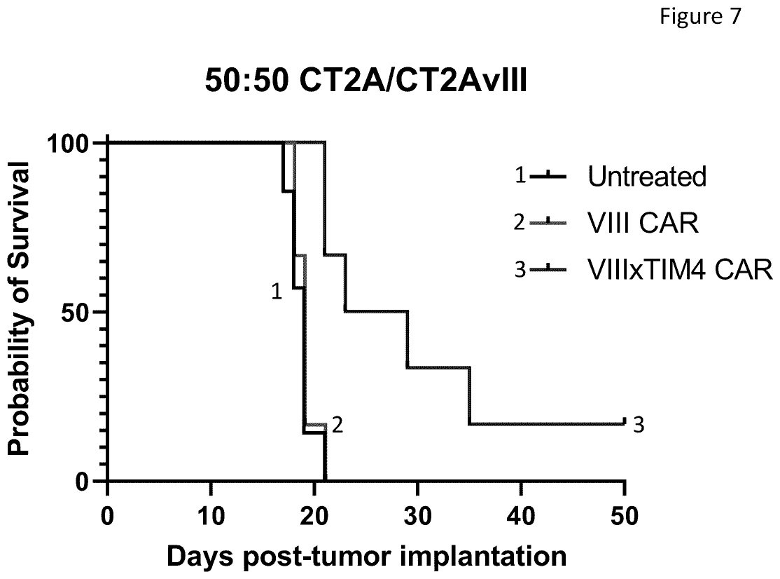

Data shown herein provides examples of the combination of heterologous species comprising recombinant mouse sequences along with human T cells. FIG. 7 shows a fully mouse recombinant TIM-4 VIII CAR vector in mouse T cells. This in vivo experiment shows that recombinant TIM-4 secreted by VIII CAR extends the survival of mice with heterologous tumors with no lymphodepletion reconditioning. FIG. 7 demonstrates that these same results are achieved when the recombinant TIM-4 fusion protein, and or CAR are of the same species as that to be treated. That is mouse recombinant TIM-4 together with mouse VIII CAR and mouse T cells results in an increase in survival similar or better to that of FIG. 6, comprising human T cells with mouse recombinant TIM-4 and VIIICAR. Together these data suggest that similar results can be expected in a fully human system, including those that include SEQ ID NOs: 3, 5, 17 and 10.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

| | Full length | Components |
|---|---|---|
| | SEQ ID NO: | SEQ ID NO: |
| Recombinant human Tim- | 1 | 2-SS |
| 4 fusion protein | | 3-hTim-4 |
| | | 4-linker |
| | | 5-hCD3 |
| Recombinant mouse Tim- | 6 | 2-SS |
| 4 fusion protein | | 7-mTim-4 |
| | | 4-linker |
| | | 8-mCD3 |
| H/M EGFRvIII CAR | 9 | 10-hEGFRvIII |
| Human EGFRVIII CAR | | 11-P2A |
| mTIM4 x hCD3 | | 12-mTIM4xhCD3 |
| | | (also 7 & 5) |
| M/M EGFRvIII CAR | 13 | 14-mEGFRvIII |
| Mouse EGFRvIII CAR | | 11-P2A |
| mTIM4 x mCD3 | | 15-mTIM4xmCD3 |
| | | (also 7, 4, 8) |
| H/M D2C7 CAR | 16 | 17-hD2C7 |
| Human D2C7 CAR | | 11-P2A |
| mTim4 x hCD3 | | 12-mTIM4xhCD3 |
| | | (also 7 & 5) |
| M/M D2C7 CAR | 18 | 19-mD2C7 |
| Mouse D2C7 CAR | | 11-P2A |
| mTim4 x mCD3 | | 15-mTIM4xmCD3 |
| | | (also 7, 4, 8) |
| H/H EGFRvIII CAR | 20 | 10-hEGFRvIII |
| Human EGFRvIII CAR | | 11-P2A |
| hTim4 x hCD3 | | 2-SS |
| | | 3-hTim-4 |
| | | 4-linker |
| | | 5-hCD3 |
| H/H D2C7 CAR | 21 | 17-hD2C7 |
| Human D2C7 CAR | | 11-P2A |
| hTim4 x hCD3 | | 2-SS |
| | | 3-hTim-4 |
| | | 4-linker |
| | | 5-hCD3 |

1. A TIM-4 fusion protein comprising a TIM-4 domain linked to a single chain variable fragment (scFv) specific for CD3, wherein the TIM-4 domain comprises SEQ ID NO: 3 or a sequence having 95% identity to SEQ ID NO: 3, and wherein the scFv specific for CD3 comprises complementarity determining regions (CDR) of SEQ ID NOs: 22-27 or sequences with at least 95% identity to SEQ ID NO: 22-27.

2. The fusion protein of claim 1, wherein the scFV specific for CD3 comprises SEQ ID NO: 5 or a sequence with at least 95% identity to SEQ ID NO: 5.

3. The fusion protein of claim 1, further comprising an N-terminal signal sequence, wherein the N-terminal signal sequence includes a signal sequence of SEQ ID NO: 2 or sequences with at least 95% identity to SEQ ID NO: 2 and wherein the TIM-4 domain is linked to the scFv specific for CD3 via a linker peptide.

4.-6. (canceled)

7. The fusion protein of claim 1, additionally comprising a tag, wherein the tag can be used for purification, tracking or imaging the fusion protein.

8. (canceled)

9. The fusion protein of claim 1, comprising SEQ ID NO: 1 or sequences with at least 90% identity to SEQ ID NO: 1.

10.-13. (canceled)

14. A pharmaceutical composition comprising a recombinant TIM-4 fusion protein of claim 1 and a pharmaceutically acceptable excipient, carrier and/or diluent.

15. A construct comprising a promoter operably linked to a polynucleotide sequence encoding the TIM-4 fusion protein of claim 1.

16. (canceled)

17. The construct of claim 15, additionally comprising a sequence encoding a chimeric antigen receptor (CAR), the CAR comprising an extracellular domain, a transmembrane domain, and an intracellular signaling domain wherein the CAR and the TIM-4 fusion protein are connected via a self-cleavage site.

18. The construct of claim 17, wherein the CAR comprises an extracellular domain comprising a scFv of SEQ ID NO: 10 or sequences with at least 95% identity to SEQ ID NO: 10 or a scFv of SEQ ID NO: 17 or sequences with at least 95% identity to SEQ ID NO: 17.

19.-22. (canceled)

23. The construct of claim 15, wherein the construct is included in a lentiviral, retroviral or AAV vector.

24. A chimeric antigen receptor (CAR)-T cell comprising the construct of claim 15 wherein the construct comprises:
a) a polynucleotide encoding a EGFR VIII CAR of SEQ ID NO: 10, a self-cleavage site, a signal sequence, a TIM-4 domain of SEQ ID NO: 3, a linker, and an scFv specific for CD3 of SEQ ID NO: 22-27 or wherein the construct comprises a polynucleotide encoding a polypeptide with at least 95% identity to SEQ ID NO: 10, 3, 22, 24, 25, 26 and 27; or
b) a polynucleotide encoding a EGFR VIII CAR of SEQ ID NO: 10, a self-cleavage site, a signal sequence, a TIM-4 domain of SEQ ID NO: 3, a linker and an scFv specific for CD3 of SEQ ID NO: 5 or wherein the construct comprises a polynucleotide encoding a polypeptide with at least 95% identity to SEQ ID NO: 10, 3 and 5; or
c) a polynucleotide encoding a D2C7 CAR of SEQ ID NO: 17, a self-cleavage site, a signal sequence, a TIM-4 domain of SEQ ID NO: 3, a linker and an scFv specific for CD3 of SEQ ID NO: 22-27 or wherein the construct comprises a polynucleotide encoding a polypeptide with at least 95% identity to SEQ ID NO 17, 3, 22, 23, 24, 25, 26 and 27; or
d) a polynucleotide encoding a D2C7 CAR of SEQ ID NO: 17, a self-cleavage site, a signal sequence, a TIM-4 domain of SEQ ID NO: 3, a linker and an scFv specific for CD3 of SEQ ID NO: 5 or wherein the construct comprises a polynucleotide encoding a polypeptide with at least 95% identity to SEQ ID NO 17, 3 and 5.

25.-28. (canceled)

29. The CAR-T cell of claim 24, wherein the self-cleavage site comprises SEQ ID NO: 11 or a sequence with at least 95% sequence identity to SEQ ID NO: 11, the signal sequence comprises SEQ ID NO: 2 or a sequence with at least 95% sequence identity to SEQ ID NO: 2 and the linker comprises SEQ ID NO: 4.

30.-35. (canceled)

36. A CAR-T cell comprising a polynucleotide encoding SEQ ID NO: 20 or a sequence with at least 95% sequence identity to SEQ ID NO: 20 or a polynucleotide encoding SEQ ID NO: 21 or a sequence with at least 95% sequence identity to SEQ ID NO: 21.

37. (canceled)

38. A method for treating cancer, the method comprising administering a therapeutically effective amount of a recombinant TIM-4 fusion protein of claim 1 and a pharmaceutically acceptable excipient, carrier and/or diluent.

39. The method of claim 38, additionally comprising administering T lymphocytes, wherein T lymphocytes are activated upon binding to the TIM-4 fusion protein.

40. (canceled)

41. (canceled)

42. A method for treating cancer, the method comprising administering a therapeutically effective amount of the CAR-T cell of claim 24 and a pharmaceutically acceptable excipient, carrier and/or diluent.

43. (canceled)

44. The method of claim 38, wherein the cancer is an EGFR-associated cancer or a PS-associated cancer.

45.-47. (canceled)

48. A method of diagnosing cancer, the method comprising administering the fusion protein of claim 7, detecting the presence or accumulation of a tag in a subject suspected of having cancer, wherein the tag allows for in vivo detection of the cancer.

49. (canceled)

50. (canceled)

51. A method of inducing a T-cell response jai in a subject suffering from cancer, the method comprising administering to the subject a therapeutically effective amount of the pharmaceutical composition of claim 14, wherein an antitumor response to the cancer is induced.

52. A method of inducing a T-cell response in a subject suffering from cancer, the method comprising administering to the subject a therapeutically effective amount of the CAR-T cells of claim 24, wherein an antitumor response to the cancer is induced.


SEQUENCE LISTING (28 sequences)

SEQ ID NO: 1 - protein, 555 aa, synthetic construct
MKWVTFISLLFLFSSAYSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 2 - protein, 18 aa, synthetic construct
MKWVTFISLLFLFSSAYS

SEQ ID NO: 3 - protein, 291 aa, synthetic construct
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQL

SEQ ID NO: 4 - protein, 5 aa, synthetic construct
GGGGS

SEQ ID NO: 5 - protein, 241 aa, synthetic construct
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 6 - protein, 518 aa, synthetic construct
MKWVTFISLLFLFSSAYSASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 7 - protein, 257 aa, synthetic construct
ASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 8 - protein, 238 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 9 - protein, 1090 aa, synthetic construct
MVLLVTSLLLCELPHPAFLLIPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMKWVTFISLLFLFSSAYSASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 10 - protein, 547 aa, synthetic construct
MVLLVTSLLLCELPHPAFLLIPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 22 aa, synthetic construct
GSGATNFSLLKQAGDVEENPGP

SEQ ID NO: 12 - protein, 521 aa, synthetic construct
MKWVTFISLLFLFSSAYSASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 13 - protein, 1078 aa, synthetic construct
MVLLVTSLLLCELPHPAFLLIPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRLEGSGATNFSLLKQAGDVEENPGPMKWVTFISLLFLFSSAYSASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 14 - protein, 538 aa, synthetic construct
MVLLVTSLLLCELPHPAFLLIPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRLE

SEQ ID NO: 15 - protein, 518 aa, synthetic construct; identical to SEQ ID NO: 6

SEQ ID NO: 16 - protein, 1092 aa, synthetic construct
MVLLVTSLLLCELPHPAFLLIPEVHLQQSGPELEKPGASVKISCKASGYSFTGYNMNWVKQSNGKSLEWIGNIDPYYGDTDYDQKFKGKATLTADKSSNTVYMQLQSLTSEDSAVYYCARGAHRDYYAMDYWGQGTSVTVSSGGGGSGGGGSGGGGSDIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGGGTKLEKKAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMKWVTFISLLFLFSSAYSASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 17 - protein, 549 aa, synthetic construct
MVLLVTSLLLCELPHPAFLLIPEVHLQQSGPELEKPGASVKISCKASGYSFTGYNMNWVKQSNGKSLEWIGNIDPYYGDTDYDQKFKGKATLTADKSSNTVYMQLQSLTSEDSAVYYCARGAHRDYYAMDYWGQGTSVTVSSGGGGSGGGGSGGGGSDIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGGGTKLEKKAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 1078 aa, synthetic construct
MVLLVTSLLLCELPHPAFLLIPEVHLQQSGPELEKPGASVKISCKASGYSFTGYNMNWVKQSNGKSLEWIGNIDPYYGDTDYDQKFKGKATLTADKSSNTVYMQLQSLTSEDSAVYYCARGAHRDYYAMDYWGQGTSVTVSSGGGGSGGGGSGGGGSDIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGGGTKLEKKAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGATNFSLLKQAGDVEENPGPMKWVTFISLLFLFSSAYSASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 19 - protein, 538 aa, synthetic construct
MVLLVTSLLLCELPHPAFLLIPEVHLQQSGPELEKPGASVKISCKASGYSFTGYNMNWVKQSNGKSLEWIGNIDPYYGDTDYDQKFKGKATLTADKSSNTVYMQLQSLTSEDSAVYYCARGAHRDYYAMDYWGQGTSVTVSSGGGGSGGGGSGGGGSDIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGGGTKLEKKAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 20 - protein, 1124 aa, synthetic construct
MVLLVTSLLLCELPHPAFLLIPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMKWVTFISLLFLFSSAYSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 21 - protein, 1126 aa, synthetic construct
MVLLVTSLLLCELPHPAFLLIPEVHLQQSGPELEKPGASVKISCKASGYSFTGYNMNWVKQSNGKSLEWIGNIDPYYGDTDYDQKFKGKATLTADKSSNTVYMQLQSLTSEDSAVYYCARGAHRDYYAMDYWGQGTSVTVSSGGGGSGGGGSGGGGSDIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGGGTKLEKKAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMKWVTFISLLFLFSSAYSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 22 - protein, 5 aa, synthetic construct
GYGMH

SEQ ID NO: 23 - protein, 17 aa, synthetic construct
VIWYDGSKKYYVDSVKG

SEQ ID NO: 24 - protein, 9 aa, synthetic construct
QMGYWHFDL

SEQ ID NO: 25 - protein, 11 aa, synthetic construct
RASQSVSSYLA

SEQ ID NO: 26 - protein, 7 aa, synthetic construct
DASNRAT

SEQ ID NO: 27 - protein, 10 aa, synthetic construct
QQRSNWPPLT

SEQ ID NO: 28 - protein, 6 aa, synthetic construct
HHHHHH